(19) United States
(12) Patent Application Publication
(10) Pub. No.: US 2022/0401352 A1
(43) Pub. Date: Dec. 22, 2022

(54) A COSMETIC COMPOSITION

(71) Applicants: Conopco, Inc., d/b/a UNILEVER, Englewood Cliffs, NJ (US); UNILEVER GLOBAL IP LIMITED, Wirral, Merseryside (GB)

(72) Inventors: Xuelan GU, Shanghai (CN); Dandan HUANG, Shanghai (CN)

(21) Appl. No.: 17/778,365

(22) PCT Filed: Nov. 4, 2020
(86) PCT No.: PCT/EP2020/080918

§ 371 Date: May 19, 2022

(30) Foreign Application Priority Data
Nov. 21, 2019 (CN) PCT/CN2019/120030
Dec. 17, 2019 (EP) 19216815.1

(51) Int. Cl. A61K 8/9789; A61K 8/49; A61Q 5/12; A61Q 5/02; A61Q 5/00

(52) U.S. Cl. CPC A61K 8/9789; A61K 8/4926; A61Q 5/12; A61Q 5/02; A61Q 5/006

(57) ABSTRACT

This invention relates to a cosmetic composition, especially one which provides synergistic anti-dandruff efficacy. This is achieved through a judicious combination of anti-dandruff agent piroctone olamine and a willow bark extract. Disclosed is a cosmetic composition comprising: (i) piroctone olamine: (ii) a willow bark extract; and (iii) a cosmetically acceptable carrier, wherein the ratio of the amount of said willow bark extract to that of said piroctone olamine is at least 1:2 parts by weight; wherein said willow bark extract is extracted from salix alba, salix glandulosa, salix purpurea or salix caroliniana.

### FIELD OF THE INVENTION

This invention relates to a cosmetic composition. The invention more particularly relates to a cosmetic composition e.g. those for care of hair, which provides anti-inflammatory efficacy.

### BACKGROUND OF THE INVENTION

Inflammation, a complicated biological host response to harmful stimuli, is a mechanism by which the host removes the stimuli and initiates the healing process for self-protection. The innate immune system for a host is the first line of defence against invading organisms in a non-specific manner. Dysregulated inflammation may cause various personal care problems including dandruff (on scalp/hair) and eczema/acnes (on skin). To assist the host organism (e.g. the human or animal) a few anti-inflammatory agents either through topical application or through oral consumption have been developed and used to mitigate the above problems.

Dandruff is an issue that affects many people globally. The condition is manifested by the shedding of clumps of dead skin cells from the scalp. These are white in colour and provide an aesthetically displeasing appearance. A factor that contributes to dandruff are certain species of the Malassezia yeasts. To combat these, anti-dandruff products have been developed in the form of hair cleansing shampoos. An example of a known anti-dandruff shampoo comprises sodium lauryl ether sulfate (an ethoxylated anionic surfactant) in combination with an anti-dandruff agent. Typical anti-dandruff agents used in hair care are metal pyrithione e.g. zinc pyrithione (ZPTO), Octopirox® (piroctone olamine), azole antimicrobials (e.g. climbazole), selenium sulfide and combinations thereof. Additionally, anti-inflammatory agents have also been used in anti-dandruff products to alleviate the ill-effects of this condition.

On the skin, one of the problems experienced by many people, especially on the face, is acne. This has a displeasing cosmetic appearance. Acne, also known as Acne vulgaris, is a common skin condition that affects nearly all adolescents and adults at some time in their lives. It has a complex etiology, involving abnormal keratinization, excess sebum production, androgen function, bacterial growth, and immune hypersensitivity. Although one or more of the above processes is correlated with acne, the one triggering factor and the exact sequence of events leading to the formation of acne lesions has not been fully understood. Other factors which have been linked to acne are presence of free radicals with subsequent oxidative stress leading to cellular damage. It has been observed that acne usually occurs in areas rich in sebaceous glands like the face, neck and back. A bacteria Propionibacterium acnes (P. acnes) has also been implicated in occurrence of acne.

Acne has been treated in many ways. Most treatments take several weeks to months before a noticeable change is seen. Benzoyl peroxide which has an antibacterial effect has been used for mild cases of acne and is also believed to prevent formation of further acne. In very severe cases of acne, antibiotics like tetracycline, erythromycin and clindamycin have been used. Antibiotics are believed to work by several mechanisms, the most important being the decrease in the number of bacteria in and around the follicle. They are also thought to reduce the irritating chemicals produced by the white blood cells in the sebum, thereby reducing the inflammatory response.

Thus, inflammation is a process that is manifest on the topical surface of the human or animal body in one or all of the above described conditions. People have attempted to alleviate the symptoms of the above conditions by developing new actives as well as exploring combination of actives that exhibit synergistic anti-inflammatory benefits.

### SUMMARY OF THE INVENTION

In accordance with a first aspect of the present invention, disclosed is a cosmetic composition comprising:
(i) piroctone olamine;
(ii) a willow bark extract; and
(iii) a cosmetically acceptable carrier, wherein the ratio of amount of said willow bark extract to that of said piroctone olamine is at least 1:2 parts by weight; wherein said willow bark extract is extracted from salix alba, salix glandulosa, salix purpurea or salix caroliniana.

In accordance with a second aspect of the present invention, disclosed is a non-therapeutic method of for reducing inflammation on a topical surface of a human or animal body comprising the step of applying a composition according to the first aspect on to the desired surface.

In accordance with a third aspect of the present invention, disclosed is a composition of the first aspect for use in preventing or reducing inflammation.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated. In other words, in specifying any ranges of values, any particular upper value can be associated with any particular lower value.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

By ‘a cosmetic composition’ as used herein, is meant to include a composition for topical application to skin, hair and/or scalp of mammals, especially human beings. Such a composition is generally applied on to the desired topical surface of the body for a period of time from a few seconds to up to 24 hours. When the period of time of application is low say of the order of a few seconds to a few minutes after which the composition is rinsed off with water or wiped away, such a composition is known as a cleansing composition or a wash-off composition. On the other hand, When the composition is applied for longer period of time say from several minutes to up to 24 hours and washed off usually during the process of normal personal cleaning, such a composition is known as a leave-on composition. The composition as per the present invention includes any product applied to a human body for also improving appearance, cleansing, odor control or general aesthetics.

By “hair care composition” as used herein, is meant to include a composition for topical application to hair or scalp of mammals, especially humans. By topical is meant that the composition is applied to the external surface of the body. In the present invention this is achieved by applying the composition on the hair or scalp. Such a composition may be generally classified as leave-on or rinse off, and includes any product applied for improving the appearance, cleansing, odor control or general aesthetics of scalp and hair. The hair care composition of the present invention could be in the form of a liquid, lotion, cream, foam, scrub, gel, shampoo, conditioner, shower gel or bar. The haircare composition of the present invention is preferably a leave-on composition. Alternatively, the hair care composition of the present invention is a wash-off composition. Compositions for achieving the desired benefits by way of ingestion into the human body are excluded from the scope of the present invention.

The present invention relates to an anti-inflammatory composition. It comprises synergistic anti-inflammatory action of the piroctone olamine with a willow bark extract claimed in the present invention.

Piroctone Olamine

Piroctone Olamine is an olamine salt of the hydroxamic acid derivative piroctone. It is commonly known as piroctone ethanolamine with the trade name Octopirox©.

The piroctone olamine according to the present invention is a 1:1 compound of 1-hydroxy-4-methyl-6-(2,4,4-trimethylpentyl)-2(1H)-pyridinone with 2-aminoethanol and is also designated 1-hydroxy-4-methyl-6-(2,4,4-trimethylpentyl)-2(1H) pyridinone monoethanolamine salt. The CAS number is 68890-66-4 and the compound has the general formula (1) as below:

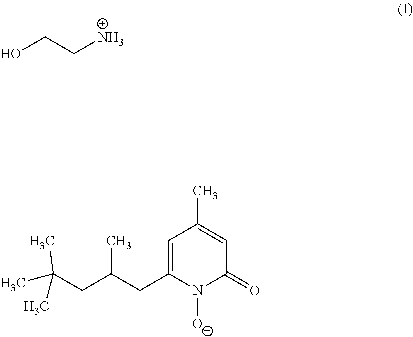

Amount of the piroctone olamine in the composition of the invention would depend on the type of the hair care composition and the precise nature of other antidandruff agents used. It is preferred that the composition comprises 0.01 to 6 wt % of said photolabile antidandruff agent, more preferably 0.1 to 5 wt %, furthermore preferably 0.5 to 3 wt % by weight of the composition.

Willow Bark Extract

The composition of the present invention comprises a willow bark extract.

Willow bark contains salicin, which is the precursor of the active ingredient in aspirin. Therefore, the willow bark extract is known as an anti-inflammatory that helps alleviate the redness, pain, and swelling that are associated with both acne and allergies and sensitivities for skin.

The willow bark extract of the present invention is extracted from salix alba, salix glandulosa, salix purpurea or salix caroliniana.

The inventors surprisingly found that the combination of piroctone olamine and willow bark extract is useful in synergistically enhancing the anti-inflammatory efficacy. This combination could provide synergistic anti-inflammatory activity when the ratio of the amount of willow bark extract to that of the piroctone olamine in the composition of the invention is at least 1:2 parts by weight, preferably at least 1:1 part by weight. It is preferred that the ratio of the amount of willow bark extract to that of the piroctone olamine is from 1:2 to 100:1 part by weight, more preferably from 1:1 to 50:1, furthermore preferably from 5:1 to 20:1, and optimally from 10:1 to 20:1 part by weight.

It is preferred that the composition of the invention comprises 0.005 to 60 wt % willow bark extract, more preferably 0.05 to 30 wt %, furthermore preferably 0.25 to 20 wt %, and optimally 0.5 to 3 wt % by weight of the composition.

The composition of the invention comprises a cosmetically acceptable carrier. According to one aspect the cosmetically acceptable carrier comprises water. According to another preferred aspect, the carrier additionally comprises a surfactant. The cosmetically acceptable carrier is such that the composition can be prepared as a wash-off or leave-on hair care composition.

It is preferred that the composition of the invention additionally comprises a glycerol. Glycerol which can be used in the present invention is variously known as glycerine, glycerin, and propane-1,2,3-triol. The glycerol can be included in the composition of the present invention at a level of from 0.5 to 60 wt %, preferably from 0.1 to 30 wt %, more preferably from 2 to 20 wt %, most preferably from 5 to 10 wt % by weight of the composition.

Without wishing to be bound by theory the inventors believe that the glycerol can improve the synergistic effect between piroctone olamine and willow bark extract. And it is also believed that willow bark extract and/or piroctone olamine can improve the function of glycerol such as enhancing skin barrier.

Hair Shampoo Composition

The composition is preferably an anti-dandruff hair care composition. The composition of the present invention is preferably a shampoo or a conditioner. It is used for preventing or alleviating the symptoms of dandruff on the scalp and/or hair.

As per an especially preferred aspect of the invention, the composition is a shampoo. The composition of the invention may comprise one or more cleansing surfactants. Surfactants are compounds which have hydrophilic and hydrophobic portions that act to reduce the surface tension of the aqueous solutions they are dissolved in. Shampoo compositions according to the invention will generally comprise one or more cleansing surfactants, which are cosmetically acceptable and suitable for topical application to the hair. The cleansing surfactant may be chosen from anionic, non-ionic, amphoteric and zwitterionic compounds and mixtures thereof.

The total amount of cleansing surfactant in a shampoo composition for use in the invention is generally from 1 to 50%, preferably from 2 to 40%, more preferably from 4 to 25% by total weight surfactant based on the total weight of the composition.

It is preferred that the shampoo composition of the invention comprises an anionic surfactant at a level of from 1 to 45% by weight of the total composition.

Non-limiting examples cleansing surfactants include anionic cleansing surfactants including; alkyl sulphates, alkyl ether sulphates, alkaryl sulphonates, N-alkyl sarcosinates, alkyl phosphates, alkyl ether phosphates, acyl amino acid based surfactants, alkyl ether carboxylic acids, acyl taurates, acyl glutamates, alkyl glycinates and salts thereof, especially their sodium, magnesium, ammonium and mono-, di- and triethanolamine salts. The alkyl and acyl groups in the preceding list generally contain from 8 to 18, preferably from 10 to 16 carbon atoms and may be unsaturated. The alkyl ether sulphates, alkyl ether phosphates and alkyl ether carboxylic acids and salts thereof may contain from 1 to 20 ethylene oxide or propylene oxide units per molecule.

Further non-limiting examples of cleansing surfactants may include non-ionic cleansing surfactants including; aliphatic (C₈-C₁₈) primary or secondary linear or branched chain alcohols with alkylene oxides, usually ethylene oxide and generally having from 6 to 30 ethylene oxide groups. Other representative cleansing surfactants include mono- or di-alkyl alkanolamides (examples include coco mono-ethanolamide and coco mono-isopropanolamide) and alkyl polyglycosides (APGs). Suitable alkyl polyglycosides for use in the invention are commercially available and include for example those materials identified as: Plantapon 1200 and Plantapon 2000 ex BASF. Other sugar-derived surfactants, which can be included in compositions for use in the invention include the C₁₀-C₁₈ N-alkyl (C₁-C₆) polyhydroxy fatty acid amides, such as the C₁₂-C₁₈ N-methyl glucamides, as described for example in WO 92 06154 and U.S. Pat. No. 5,194,639, and the N-alkoxy polyhydroxy fatty acid amides, such as C₁₀-C₁₈ N-(3-methoxypropyl) glucamide.

Additional non-limiting examples of cleansing surfactants include amphoteric or zwitterionic cleansing surfactants including; alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, wherein the alkyl and acyl groups have from 8 to 19 carbon atoms.

Typical cleansing surfactants for use in shampoo compositions for use in the invention include sodium oleyl succinate, ammonium lauryl sulphosuccinate, sodium lauryl sulphate, sodium lauryl ether sulphate, sodium lauryl ether sulphosuccinate, ammonium lauryl sulphate, ammonium lauryl ether sulphate, sodium cocoyl isethionate, sodium lauryl isethionate, lauryl ether carboxylic acid and sodium N-lauryl sarcosinate, sodium pareth sulphate, cocodimethyl sulphopropyl betaine, lauryl betaine, coco betaine, cocamidopropyl betaine, sodium cocoamphoacetate.

Preferred cleansing surfactants are sodium lauryl sulphate, sodium lauryl ether sulphate, sodium lauryl ether sulphosuccinate, ammonium lauryl sulphate, ammonium lauryl ether sulphate, sodium cocoyl isethionate and lauryl ether carboxylic acid, coco betaine, cocamidopropyl betaine, sodium cocoamphoacetate.

Mixtures of any of the foregoing anionic, non-ionic and amphoteric cleansing surfactants may also be suitable, preferably where the primary to secondary surfactant ratio is between 1:1-10:1, more preferably 2:1-9:1 and most preferably 3:1-8:1, based on the inclusion weight of the cleansing surfactant in the shampoo composition.

Suspending Agent

Preferably the composition of the invention further comprises a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the composition. Polyacrylic acid is available commercially as Carbopol 420, Carbopol 488 or Carbopol 493. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. All Carbopol (trademark) materials are available from Goodrich.

Suitable cross-linked polymers of acrylic acid and acrylate esters are Pemulen® TR1 or Pemulen® TR2. A suitable heteropolysaccharide gum is xanthan gum, for example that available as Kelzan® mu.

Mixtures of any of the above suspending agents may be used. Preferred is a mixture of cross-linked polymer of acrylic acid and crystalline long chain acyl derivative.

Suspending agent, if included, will generally be present in a shampoo composition of the invention at levels of from 0.1 to 10%, preferably from 0.5 to 6%, more preferably from 0.5 to 4% by total weight of suspending agent based on the total weight of the composition.

A composition of the invention may contain other ingredients for enhancing performance and/or consumer acceptability. Such ingredients include fragrance, dyes and pigments, pH adjusting agents, pearlescers or opacifiers, viscosity modifiers, preservatives, and natural hair nutrients such as botanicals, fruit extracts, sugar derivatives and amino acids.

The composition of the invention is preferably aqueous based. It preferably comprises high amounts of water preferably from 70 to 95% by weight of the composition.

Cationic Deposition Polymer

The composition of the invention may further comprise a cationic deposition polymer. Suitable cationic polymers may be homopolymers which are cationically substituted or may be formed from two or more types of monomers. The weight average (M_{w}) molecular weight of the polymers will generally be between 100 000 and 2 million daltons. The polymers will have cationic nitrogen containing groups such as quaternary ammonium or protonated amino groups, or a mixture thereof. If the molecular weight of the polymer is too low, then the conditioning effect is poor. If too high, then there may be problems of high extensional viscosity leading to stringiness of the composition when it is poured.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give polymers having a cationic charge density in the required range, which is generally from 0.2 to 3.0 meq/gm. The cationic charge density of the polymer is suitably determined via the Kjeldahl method as described in the US Pharmacopoeia under chemical tests for nitrogen determination.

Suitable cationic polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerised in the amine form and then converted to ammonium by quaternization.

The cationic polymers can comprise mixtures of monomer units derived from amine- and/or quaternary ammonium-substituted monomer and/or compatible spacer monomers.

Suitable (non-limiting examples of) cationic polymers include:
cationic diallyl quaternary ammonium-containing polymers including, for example, dimethyldiallylammonium chloride homopolymer and copolymers of acrylamide and dimethyldiallylammonium chloride, referred to in the industry (CTFA) as Polyquaternium 6 and Polyquaternium 7, respectively;
mineral acid salts of amino-alkyl esters of homo- and co-polymers of unsaturated carboxylic acids having from 3 to 5 carbon atoms, (as described in U.S. Pat. No. 4,009,256);
cationic polyacrylamides (as described in WO95/22311).

Other cationic polymers that can be used include cationic polysaccharide polymers, such as cationic cellulose derivatives, cationic starch derivatives, and cationic guar gum derivatives.

Cationic polysaccharide polymers suitable for use in compositions for use in the invention include monomers of the formula:

A—O—[R—N⁺(R¹)(R²)(R³)X⁻],

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from the Amerchol Corporation, for instance under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581). Examples of such materials include the polymer LR and JR series from Dow, generally referred to in the industry (CTFA) as Polyquaternium 10.

A particularly suitable type of cationic polysaccharide polymer that can be used is a cationic guar gum derivative, such as guar hydroxypropyltrimethylammonium chloride (commercially available from Rhodia in their JAGUAR trademark series). Examples of such materials are JAGUAR C13S, JAGUAR C14 and JAGUAR C17.

Mixtures of any of the above cationic polymers may be used.

Cationic polymer will generally be present in a shampoo composition for use in the invention at levels of from 0.01 to 5%, preferably from 0.02 to 1%, more preferably from 0.05 to 0.8% by total weight of cationic polymer based on the total weight of the composition.

Hair Conditioner

When conditioning benefits are to be delivered through the composition of the invention the composition is called a hair conditioner. Typically, the most popular conditioning agents used in hair care compositions are water-insoluble oily materials such as mineral oils, naturally occurring oils such as triglycerides and silicone polymers. Conditioning benefit is achieved by the oily material being deposited onto the hair resulting in the formation of a film, which makes the hair easier to comb when wet and more manageable when dry. An especially useful conditioning agent is a silicone compound, preferably a non-volatile silicone compound. Advantageously compositions herein may include one or more silicones. The silicones are conditioning agents found in dispersed or suspended particulate form. They are intended to deposit onto hair remaining behind after rinsing of the hair with water. Suitable silicone oils may include polyalkyl siloxanes, polyaryl siloxanes, polyalkylaryl siloxanes, polyether siloxane copolymers and mixtures thereof. Amino silicones are often formulated with shampoo compositions. Amino silicones are silicones containing at least one primary amine, secondary amine, tertiary amine or a quaternary ammonium group. High molecular weight silicone gums can also be utilized. Another useful type are the crosslinked silicone elastomers such as Dimethicone/Vinyl/Dimethicone Crosspolymers (e.g. Dow Corning 9040 and 9041).

Amounts of the silicone in compositions where present may range from about 0.1 to about 10 wt. %, preferably from about 0.1 to about 8 wt. %, more preferably from about 0.3 to about 5 wt. % by weight of the hair care compositions.

The pH of the composition is preferably equal to or higher than 4.0, more preferably in the range of 5.0 to 7.0.

The hair conditioning composition usually comprises conditioning surfactants selected from cationic surfactants, used singly or in admixture. Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese. Another particularly useful cationic surfactant for use in conditioners according to the invention is behenyltrimethylammonium chloride, available commercially, for example as GENAMIN® KDMP, ex Clariant. Yet another preferred cationic surfactant is stearamidopropyl dimethylamine.

The most preferred cationic surfactants for use in the composition are stearamidopropyl dimethylamine, behentrimonium chloride, or stearyl trimethyl ammonium chloride. In conditioners of the invention, the level of cationic surfactant will generally range from 0.1% to 5%, preferably 0.5 to 2.5% by weight of the composition.

Hair conditioning compositions of the invention preferably may also additionally comprise a fatty alcohol. The combined use of fatty alcohols and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is also advantageous in that they contribute to the overall conditioning properties of compositions of the invention.

The level of fatty alcohol in conditioners of the invention will generally range from 0.5 to 10%, preferably from 0.1% to 8%, more preferably from 0.2% to 7%, most preferably from 0.3% to 6% by weight of the composition. The weight ratio of cationic surfactant to fatty alcohol is suitably from 1:1 to 1:10, more preferably from 1:1.5 to 1:8, optimally from 1:2 to 1:5.

Method and Use

The invention also provides for a non-therapeutic method of reducing inflammation on a topical surface of a human or animal body comprising the step of applying the composition of the invention on to the desired surface. The method is cosmetic in nature. The invention also provides for a non-therapeutic method of preventing or alleviating the symptoms of dandruff on the scalp and/or hair comprising the step of applying the composition of the invention on to scalp and/or hair. The method is cosmetic in nature.

The invention also provides for a composition of the invention for use to reduce or prevent inflammation. The invention provides for a composition of the invention for use in preventing or alleviating the symptoms of dandruff on the scalp and/or hair.

The present invention also provides a combination of piroctone olamine and willow bark extract for use to reduce or prevent inflammation; wherein ratio of the amount of said willow bark extract to that of said piroctone olamine is at least 1:2 parts by weight; wherein said willow bark extract is extracted from salix alba, salix glandulosa, salix purpurea or salix caroliniana.

The present invention also provides a combination of piroctone olamine and willow bark extract for use in preventing or alleviating the symptoms of dandruff on the scalp and/or hair; wherein ratio of the amount of said willow bark extract to that of said piroctone olamine is at least 1:2 parts by weight; wherein said willow bark extract is extracted from salix alba, salix glandulosa, salix purpurea or salix caroliniana.

The present invention provides a topical composition comprising piroctone olamine and willow bark extract for use in the treatment of dandruff; wherein ratio of the amount of said willow bark extract to that of said piroctone olamine is at least 1:2 parts by weight; wherein said willow bark extract is extracted from salix alba, salix glandulosa, salix purpurea or salix caroliniana.

The invention will now be illustrated with reference to the following non-limiting Examples.

### Examples

Willow bark extract used in the examples was the white willow bark extract (extracted from salix alba) which was purchased from Xinrui Bio-technology Limited.

Human Immune Cell Line THP-1 Invitro Assay

The following procedure was used to determine the anti-inflammation efficacy: THP1-XBlue™ (Cat No: thpx-sp, InvivoGen) cells were cultured as suspense in RPMI 1640 medium supplemented with 10% FBS, penicillin (10 U/mL)-streptomycin (10 μg/ML). Cells were differentiated in 24-well plates at the density of 5×10⁵ cells/well with 100 nM PMA for 72 hours. Cells were then co-treated with pure E. coli ipopolysaccharides (LPS) and various compositions. After 24 hours, the supernatants were collected and measured for interleukin (IL)-6 as a pro-inflammatory biomarker using enzyme-linked immunosorbent assay (ELISA). IL-6 is a cytokine, or cell-signaling protein, encoded in humans by the IL-6 gene which is both pro-inflammatory and anti-inflammatory and which stimulates the immune response to inflammation. Expression of IL-6 was calculated as the percentage to LPS-treated cells which was designated as 100%. It can be considered as no anti-inflammatory effect if the calculated result is over 100%. Cell viability was calculated as the percentage to non-treated cells which was designated as 100%. P value was analyzed by Student's t-test comparing combination groups and individual compound groups (expression of IL-6). P value <0.05 indicates synergistic effect.

The results in terms of expression of IL-6 in percentage and cell viability are given in Table 1:

TABLE 1

| | | expres- | | | | |
|---|---|---|---|---|---|---|
| | | sion | | | Cell | |
| Exam- | | of IL-6 | Std. | P | Viability | Std. |
| ples | Composition | (%) | dev | value | (%) | dev |
| A | LPS | 100 | 0 | NA | 84 | 3 |
| B | 0.15 ppm Octopirox | 76.4 | 2.1 | NA | 81 | 2 |
| C | 3 ppm salicin | 65.0 | 5.0 | NA | 86 | 2 |
| D | 0.03 ppm willow | 79.3 | 4.0 | NA | 85 | 3 |
| | bark extract | | | | | |
| E | 0.075 ppm willow | 87.8 | 1.1 | NA | 84 | 3 |
| | bark extract | | | | | |
| F | 3 ppm willow bark | 82.5 | 3.3 | NA | 80 | 3 |
| | extract | | | | | |
| G | 3 ppm salicin + 0.15 | 59.5 | 7.1 | 0.37 | 80 | 1 |
| | ppm Octopirox | | | | | |
| | (Ratio: 20:1 by | | | | | |
| | weight) | | | | | |
| H | 0.03 ppm willow | 63.3 | 1.3 | 0.13 | 88 | 2 |
| | bark extract + 0.15 | | | | | |
| | ppm Octopirox | | | | | |
| | (Ratio: 1:5 by | | | | | |
| | weight) | | | | | |
| 1 | 0.075 ppm willow | 61.0 | 3.7 | 0.03 | 85 | 1 |
| | bark extract + 0.15 | | | | | |
| | ppm Octopirox | | | | | |
| | (Ratio: 1:2 by | | | | | |
| | weight) | | | | | |
| 2 | 3 ppm willow bark | 61.2 | 6.0 | 0.03 | 82 | 8 |
| | extract + 0.15 ppm | | | | | |
| | Octopirox (Ratio: | | | | | |
| | 20:1 by weight) | | | | | |

The data in Table 1 indicates that compositions as per the invention (Examples 1 and 2) are capable of delivering synergistic anti-inflammatory efficacy under the test conditions disclosed earlier without impairing the cell viability, while compositions outside the invention (Examples G and H) do not exhibit any synergistic effect (P value >0.05). It can be also observed that when the ratio of the willow bark extract to that of the piroctone olamine of is further increased, the synergistic anti-inflammatory efficacy of the combination is maintained without impairing the cell viability (Examples 1 and 2). It is also surprisingly observed that salicin at same dosage (Example G) does not exhibit synergistic effect with Octopirox while the willow bark extract (Example 2) can.

All the experiments disclosed hereinabove were conducted under in vitro conditions to ascertain whether the combination of anti-inflammatory actives was synergistic, additive or antagonistic vis-a-vis their individual activity. As far as the experiments were concerned, the concentrations of the ingredients were chosen to fall within the allowable limits permitted by the concerned tests and in which it was possible to record the technical effects. Therefore, the concentrations at which the tests were conducted might not appear to fall within the range in which such ingredients are generally used in cosmetic compositions (usually in wt %).

It is to be understood that the experiments described above were conducted in an in vitro assay to evaluate the synergistic anti-inflammatory behaviour. It is expected that the concentrations to be actually used to prepare a composition for topical use would be vastly different.

The composition may be formulated as an emulsion or a gel with very many additional ingredients which affect the concentration of the desired actives in the oil phase and in the water phase which could be very different. They may also have very different physical and hydrodynamic properties like partition coefficients, diffusional rates, convective transport rates, rheological properties etc. Therefore, it is expected that the concentrations to be used when formulated as a composition would be very different from that at the cellular level, at which the experiments were carried out, usually orders of magnitude higher.

1. A cosmetic composition comprising:
(i) piroctone olamine;
(ii) a willow bark extract; and
(iii) a cosmetically acceptable carrier; wherein ratio of the amount of said willow bark extract to that of said piroctone olamine is at least 1:2 parts by weight; wherein said willow bark extract is extracted from salix alba, salix glandulosa, salix purpurea, or salix caroliniana.

2. The composition as claimed in claim 1 wherein ratio of the amount of said willow bark extract to that of said piroctone olamine is from 1:2 to 100:1 part by weight.

3. The composition as claimed in claim 1 wherein said composition comprises 0.01 to 6 wt % piroctone olamine.

4. The composition as claimed in claim 1 wherein said composition comprises 0.005 to 60 wt % willow bark extract.

5. The composition as claimed in claim 1 wherein said composition additionally comprises a surfactant.

6. The composition as claimed in claim 1 wherein said composition is a wash-off or leave-on hair care composition.

7. The composition as claimed in claim 6 wherein said composition is a shampoo or a conditioner.

8. The composition as claimed in claim 7 wherein said composition is a shampoo and comprises an anionic surfactant at a level of from 1 to 45% by weight of the total composition.

9. The composition as claimed in claim 1 wherein said composition additionally comprises a cationic deposition polymer.

10. A non-therapeutic method of reducing inflammation on a topical surface of a human or animal body comprising the step of applying the composition as claimed in claim 1 to the desired surface.

11. A non-therapeutic method of preventing or alleviating the symptoms of dandruff on the scalp and/or hair comprising the step of applying a composition as claimed in claim 1 on to scalp and/or hair.

12. (canceled)

13. (canceled)

14. A method of treating dandruff comprising the step of applying to scalp and/or hair a topical composition comprising piroctone olamine and willow bark extract; wherein ratio of the amount of said willow bark extract to that of said piroctone olamine is at least 1:2 parts by weight; wherein said willow bark extract is extracted from salix alba, salix glandulosa, salix purpurea, or salix caroliniana.

15. The method as claimed in claim 15, wherein the amount of said willow bark extract to that of said piroctone olamine is from 1:2 to 100:1 part by weight.

16. The method as claimed in claim 15, wherein said composition comprises 0.01 to 6 wt % piroctone olamine.

17. The method as claimed in claim 15, wherein said composition comprises 0.005 to 60 wt % willow bark extract.

18. The composition as claimed in claim 3, wherein said composition comprises 0.005 to 60 wt % willow bark extract.
